SEQUENCE LISTING (9 sequences)

SEQ ID NO: 1 - DNA, 16 bp, homo sapiens
atggacttagcattcg

SEQ ID NO: 2 - DNA, 12 bp, homo sapiens
atggacttagca

SEQ ID NO: 3 - DNA, 12 bp, homo sapiens
tggacttagcat

SEQ ID NO: 4 - DNA, 12 bp, homo sapiens
ggacttagcatt

SEQ ID NO: 5 - DNA, 12 bp, homo sapiens
gacttagcattc

SEQ ID NO: 6 - DNA, 12 bp, homo sapiens
acttagcattcg

SEQ ID NO: 7 - DNA, 25 bp, homo sapiens
atcatcgtagctggtcagtgtatcc

SEQ ID NO: 8 - DNA, 25 bp, homo sapiens
atcatcgtagctggtcagtgtatcc

SEQ ID NO: 9 - DNA, 35 bp, homo sapiens
atcatcgtagctggtcagtgtatccaaaaaaaaaa

(19) United States
(12) Patent Application Publication
(10) Pub. No.: US 2005/0095596 A1
(43) Pub. Date: May 5, 2005

(54) Methods for identifying suitable nucleic acid probe sequences for use in nucleic acid arrays

(76) Inventors: Eric M. Leproust, San Jose, CA (US); Paul K. Wolber, Los Altos, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/698,195

(22) Filed: Oct. 30, 2003

(51) Int. Cl. C12Q 1/68

(52) U.S. Cl. 435/6

(57) ABSTRACT

Methods of identifying a sequence of a probe, e.g., a biopolymeric probe, such as a nucleic acid, for use as a surface immobilized probe for a target molecule of interest, e.g., a target nucleic acid, are provided. A feature of the subject methods is that a set of candidate sequences is evaluated for full-length synthesis probability, e.g., by evaluating the candidate sequences' depurination susceptibility. The subject invention also includes algorithms for performing the subject methods recorded on a computer readable medium, as well as computational analysis systems that include the same. Also provided are nucleic acid arrays produced with probes having sequences identified by the subject methods, as well as methods for using the same.

### RELEVANT LITERATURE

U.S. Patents of interest include: U.S. Pat. Nos. 6,251,588 and 5,556,749. Also of interest is Hosaka et al., Genome Informatics (2001) 12: 449-450.

### FIELD OF THE INVENTION

The field of this invention is nucleic acid arrays.

### BACKGROUND OF THE INVENTION

Arrays of binding agents or probes, such as polypeptide and nucleic acids, have become an increasingly important tool in the biotechnology industry and related fields. These binding agent arrays, in which a plurality of probes are positioned on a solid support surface in the form of an array or pattern, find use in a variety of different fields, e.g., genomics (in sequencing by hybridization, SNP detection, differential gene expression analysis, identification of novel genes, gene mapping, finger printing, etc.) and proteomics.

In using such arrays, the surface bound probes are contacted with molecules or analytes of interest, i.e., targets, in a sample. Targets in the sample bind to the complementary probes on the substrate to form a binding complex. The pattern of binding of the targets to the probe features or spots on the substrate produces a pattern on the surface of the substrate and provides desired information about the sample. In most instances, the targets are labeled with a detectable label or reporter such as a fluorescent label, chemiluminescent label or radioactive label. The resultant binding interaction or complexes of binding pairs are then detected and read or interrogated, for example by optical means, although other methods may also be used depending on the detectable label employed. For example, laser light may be used to excite fluorescent labels bound to a target, generating a signal only in those spots on the substrate that have a target, and thus a fluorescent label, bound to a probe molecule. This pattern may then be digitally scanned for computer analysis.

Generally, in discovering or designing probes to be used in an array, a nucleic acid sequence is selected based on the particular gene of interest, where the nucleic acid sequence may be as great as about 60 or more nucleotides in length or as small as about 25 nucleotides in length or less. From the nucleic acid sequence, probes are synthesized according to various nucleic acid sequence regions, i.e., subsequences, of the nucleic acid sequence and are associated with a substrate to produce a nucleic acid array. As described above, a detectably labeled sample is contacted with the array, where targets in the sample bind to complimentary probe sequences of the array.

As is apparent, a key step in designing arrays is the selection of a specific probe or mixture of probes that may be used in the array and which maximize the chances of binding with a specific target in a sample, while at the same time minimize the time and expense involved in probe discovery and design. In practice, designing an optimized array typically involves iterating the array design one or more times to replace probes that are found to be undesirable for detecting targets of interest, either due to poor signal quality and/or cross-hybridization with sequences other than the targets of interest. Such iterations are costly and time consuming.

For example, conventional probe design may be performed experimentally or computationally, where in many instances it is performed computationally. Accordingly, probe design usually involves taking subsequences of a nucleic acid and filtering them based on certain computationally determined values such as melting temperature, self structure, homology, etc., to attempt to predict which subsequences will generate probes that will provide good signal and/or will not cross-hybridize. The subsequences that remain after the filtering process are selected to generate probes to be used in nucleic acid arrays.

There is continued interest in the development of new methods and devices for designing nucleic acid probes for use on nucleic acid arrays.

### SUMMARY OF THE INVENTION

Methods of identifying a sequence of a probe, e.g., a biopolymeric probe, such as a nucleic acid, for use as a surface immobilized probe for a target molecule of interest, e.g., a target nucleic acid, are provided. A feature of the subject methods is that a set of candidate sequences is evaluated for full-length synthesis probability, e.g., by evaluating the candidate sequences' depurination susceptibility. The subject invention also includes algorithms for performing the subject methods recorded on a computer readable medium, as well as computational analysis systems that include the same. Also provided are nucleic acid arrays produced with probes having sequences identified by the subject methods, as well as methods for using the same.

### BRIEF DESCRIPTIONS OF THE DRAWING

FIG. 1 shows an exemplary substrate carrying an array, such as may be used in the devices of the subject invention.

FIG. 2 shows an enlarged view of a portion of FIG. 1 showing spots or features.

FIG. 3 is an enlarged view of a portion of the substrate of FIG. 1.

### DEFINITIONS

In the present application, unless a contrary intention appears, the following terms refer to the indicated characteristics.

The term “polymer” means any compound that is made up of two or more monomeric units covalently bonded to each other, where the monomeric units may be the same or different, such that the polymer may be a homopolymer or a heteropolymer. Representative polymers include peptides, polysaccharides, nucleic acids and the like, where the polymers may be naturally occurring or synthetic.

The term “biopolymer” refers to a polymer of one or more types of repeating units. Biopolymers are typically found in biological systems and particularly include polysaccharides (such as carbohydrates), and peptides (which term is used to include polypeptides and proteins) and polynucleotides as well as their analogs such as those compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. For example, a “biopolymer” includes DNA (including cDNA), RNA, oligonucleotides, and PNA and other polynucleotides as described in U.S. Pat. No. 5,948,902 and references cited therein (all of which are incorporated herein by reference), regardless of the source.

The term “nucleic acid” as used herein means a polymer composed of nucleotides, e.g., deoxyribonucleotides or ribonucleotides, or compounds produced synthetically (e.g. PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” refers to a nucleotide multimer of about 10 to 100 nucleotides in length and up to 200 nucleotides in length.

The term “polynucleotide” as used herein refers to a nucleotide multimer having any number of nucleotides.

The term “biomonomer” references a single unit, which can be linked with the same or other biomonomers to form a biopolymer (for example, a single amino acid or nucleotide with two linking groups one or both of which may have removable protecting groups). The terms “biomonomer fluid” and “biopolymer fluid” reference a liquid containing either a biomonomer or biopolymer, respectively (typically in solution).

The term “monomer” as used herein refers to a chemical entity that can be covalently linked to one or more other such entities to form a polymer. Examples of “monomers” include nucleotides, amino acids, saccharides, peptides, other reactive organic molecules and the like. In general, the monomers used in conjunction with the present invention have first and second sites (e.g., C-termini and N-termini (for proteins), or 5′ and 3′ sites(for oligomers, RNA's, cDNA's, and DNA's)) suitable for binding to other like monomers by means of standard chemical reactions (e.g., condensation, nucleophilic displacement of a leaving group, or the like), and a diverse element which distinguishes a particular monomer from a different monomer of the same type (e.g., an amino acid side chain, a nucleotide base, etc.). In the art synthesis of biomolecules of this type utilize an initial substrate-bound monomer that is generally used as a building-block in a multi-step synthesis procedure to form a complete ligand, such as in the synthesis of oligonucleotides, oligopeptides, and the like.

The term “oligomer” is used herein to indicate a chemical entity that contains a plurality of monomers. As used herein, the terms “oligomer” and “polymer” are used interchangeably. Examples of oligomers and polymers include polydeoxyribonucleotides (DNA), polyribonucleotides (RNA), other polynucleotides which are C-glycosides of a purine or pyrimidine-base, -polypeptides (proteins), polysaccharides (starches, or polysugars), and other chemical entities that contain repeating units of like chemical structure.

The term “sample” as used herein relates to a material or mixture of materials, typically, although not necessarily, in fluid form, containing one or more targets, i.e., components or analytes of interest.

The terms “nucleoside” and “nucleotide” refer to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence-specific manner analogous to that of two naturally occurring polynucleotides. The terms “nucleoside” and “nucleotide” are intended to include those moieties which contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The terms “may” “optional” or “optionally” used herein interchangeably means that the subsequently described circumstance may or may not occur, so that the description includes instances where the circumstance occurs and instances where it does not.

The terms “probe”, “probe sequence”, “target probe” or “ligand” as used herein refer to a moiety made of an oligonucleotide or polynucleotide, as defined above, which contains a nucleic acid sequence complementary to a nucleic acid sequence present in a sample of interest such that the probe will specifically hybridize to the nucleic acid sequence present in the sample under appropriate conditions. The nucleic acid probes of the subject invention are typically associated with a support or substrate to provide an array of nucleic acid probes to be used in an array assay. The term “probe” or its equivalents as used herein refer to a compound that is “pre-synthesized” or obtained commercially, and then attached to the substrate or synthesized on the substrate, i.e., synthesized in situ on the substrate. The nucleic acid probes of the subject invention are produced, generated or synthesized according to probe sequences identified as suitable according to the subject invention that may or may not have been further tested or characterized.

The terms “reporter”, “label” “detectable reporter” and “detectable label” are used herein to refer to a molecule capable of detection, including, but not limited to, radioactive isotopes, fluorescers, chemiluminescers, enzymes, enzyme substrates, enzyme cofactors, enzyme inhibitors, dyes, metal ions, metal sols, other suitable detectable markers such as biotin or haptens and the like. The term “fluorescer” refers to a substance or portion thereof which is capable of exhibiting fluorescence in the detectable range. The term “cofactor” is used broadly herein to include any molecular moiety that participates in an enzymatic reaction. Particular example of labels which may be used under the invention include, but are not limited to, fluorescein, 5(6)-carboxyfluorescein, Cyanine 3 (Cy3), Cyanine 5 (Cy5), rhodamine, dansyl, umbelliferone, Texas red, luminal, NADPH, horseradish peroxidase and α,β-galactosidase.

An “array,” includes any one-dimensional, two-dimensional or substantially two-dimensional (as well as a three-dimensional) arrangement of addressable regions bearing a particular chemical moiety or moieties (such as ligands, e.g., biopolymers such as polynucleotide or oligonucleotide sequences (nucleic acids), polypeptides (e.g., proteins), carbohydrates, lipids, etc.) associated with that region. In the broadest sense, the arrays of many embodiments are arrays of polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

Any given substrate may carry one, two, four or more or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulsejets of either polynucleotide precursor units (such as monomers) in the case of in situ fabrication, or the previously obtained polynucleotide. Such methods are described in detail in, for example, the previously cited references including U.S. Pat. No. 6,242,266, U.S. Pat. No.6,232,072, U.S. Pat. No.6,180,351, U.S. Pat. No.6,171,797, U.S. Pat. No.6,323,043, U.S. patent application Ser. No. 09/302,898 filed Apr. 30, 1999 by Caren et al., and the references cited therein. These references are incorporated herein by reference. Other drop deposition methods can be used for fabrication, as previously described herein.

With respect to methods in which premade probes are immobilized on a substrate surface, immobilization of the probe to a suitable substrate may be performed using conventional techniques. See, e.g., Letsinger et al. (1975) Nucl. Acids Res. 2:773-786; Pease, A. C. et al., Proc. Nat. Acad. Sci. USA, 1994, 91:5022-5026. The surface of a substrate may be treated with an organosilane coupling agent to functionalize the surface. One exemplary organosilane coupling agent is represented by the formula RₙSiY₍₄₋ₙ₎ wherein: Y represents a hydrolyzable group, e.g., alkoxy, typically lower alkoxy, acyloxy, lower acyloxy, amine, halogen, typically chlorine, or the like; R represents a nonhydrolyzable organic radical that possesses a functionality which enables the coupling agent to bond with organic resins and polymers; and n is 1, 2 or 3, usually 1. One example of such an organosilane coupling agent is 3-glycidoxypropyltrimethoxysilane (“GOPS”), the coupling chemistry of which is well-known in the art. See, e.g., Arkins, “Silane Coupling Agent Chemistry,” Petrarch Systems Register and Review, Eds. Anderson et al. (1987). Other examples of organosilane coupling agents are (γ-aminopropyl)triethoxysilane and (γ-aminopropyl)trimethoxysilane. Still other suitable coupling agents are well known to those skilled in the art. Thus, once the organosilane coupling agent has been covalently attached to the support surface, the agent may be derivatized, if necessary, to provide for surface functional groups. In this manner, support surfaces may be coated with functional groups such as amino, carboxyl, hydroxyl, epoxy, aldehyde and the like.

Use of the above-functionalized coatings on a solid support provides a means for selectively attaching probes to the support. For example, an oligonucleotide probe formed as described above may be provided with a 5′-terminal amino group that can be reacted to form an amide bond with a surface carboxyl using carbodiimide coupling agents. 5′ attachment of the oligonucleotide may also be effected using surface hydroxyl groups activated with cyanogen bromide to react with 5′-terminal amino groups. 3′-terminal attachment of an oligonucleotide probe may be effected using, for example, a hydroxyl or protected hydroxyl surface functionality.

In situ prepared ligand arrays, e.g., nucleic acid arrays, may be characterized by having surface properties of the substrate that differ significantly between the feature and inter-feature areas. Specifically, such arrays may have high surface energy, hydrophilic features and hydrophobic, low surface energy hydrophobic interfeature regions. Whether a given region, e.g., feature or interfeature region, of a substrate has a high or low surface energy can be readily determined by determining the regions “contact angle” with water. “Contact angle” of a liquid with a surface is the acute angle measured between the edge of a drop of liquid on that surface and the surface. Contact angle measurements are well known and can be obtained by various instruments such as an FTA200 available from First Ten Angstroms, Portsmouth, Va., U.S.A. Surfaces which are more hydrophobic (which have a lower surface energy) will have higher contact angles with water or aqueous liquids than surfaces which are less hydrophobic (and therefore a higher surface energy) (for example, a hydrophobic surface may have a water drop contact angle of more than 50 degrees, or even more than 90 degrees). The contact angle of an array (sometimes referenced as the “average contact angle” or “effective contact angle”) is the average contact angle of the features of that array and the inter-feature areas. Contact angles are measured with water unless otherwise indicated.

In certain embodiments, high surface energy regions, e.g., features, may have contact angles that are less than 45 degrees, less than 20 degrees (or less than 15, 10, or 5 degrees), while low surface energy, e.g., inter-feature, areas may have contact angles greater than 80 degrees (or even greater than 90, 95, 100, 105, 110, 115, 120 or 130 degrees).

Also, instead of drop deposition methods, light directed fabrication methods may be used, as are known in the art. Inter-feature areas need not be present particularly when the arrays are made by light directed synthesis protocols.

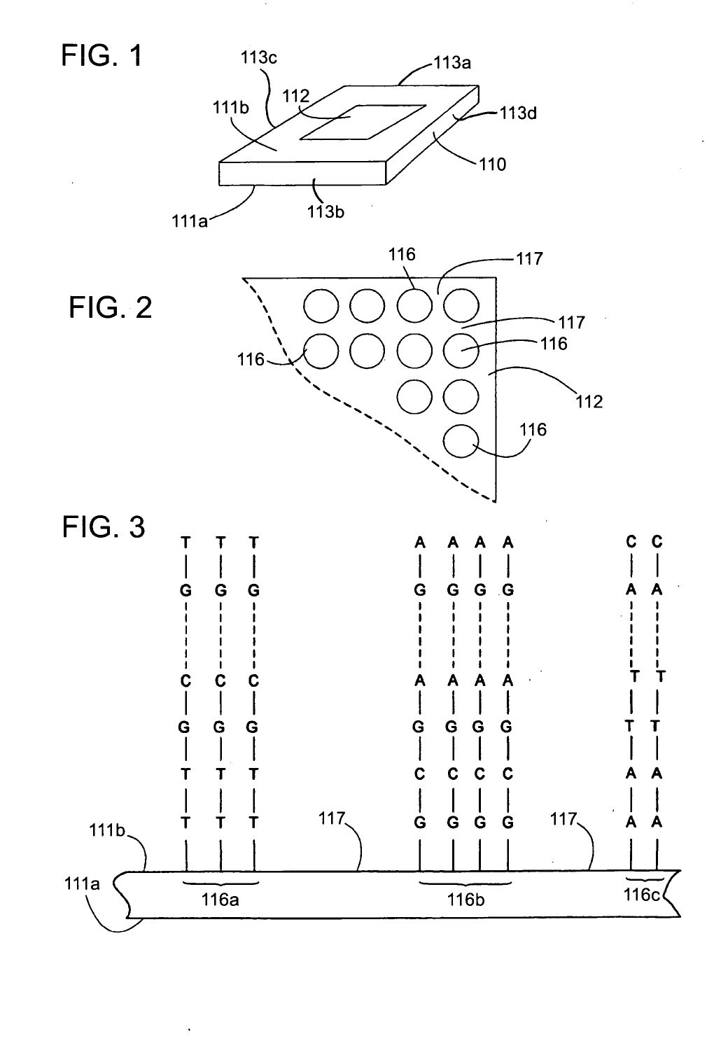

An exemplary array is shown in FIGS. 1-3, where the array shown in this representative embodiment includes a contiguous planar substrate 110 carrying an array 112 disposed on a rear surface 111b of substrate 110. It will be appreciated though, that more than one array (any of which are the same or different) may be present on rear surface 111b, with or without spacing between such arrays. That is, any given substrate may carry one, two, four or more arrays disposed on a front surface of the substrate and depending on the use of the array, any or all of the arrays may be the same or different from one another and each may contain multiple spots or features. The one or more arrays 112 usually cover only a portion of the rear surface 111b, with regions of the rear surface 111b adjacent the opposed sides 113c, 113d and leading end 113a and trailing end 113b of slide 110, not being covered by any array 112. A front surface 111a of the slide 110 does not carry any arrays 112. Each array 112 can be designed for testing against any type of sample, whether a trial sample, reference sample, a combination of them, or a known mixture of biopolymers such as polynucleotides. Substrate 110 may be of any shape, as mentioned above.

As mentioned above, array 112 contains multiple spots or features 116 of biopolymers, e.g., in the form of polynucleotides. As mentioned above, all of the features 116 may be different, or some or all could be the same. The interfeature areas 117 could be of various sizes and configurations. Each feature carries a predetermined biopolymer such as a predetermined polynucleotide (which includes the possibility of mixtures of polynucleotides). It will be understood that there may be a linker molecule (not shown) of any known types between the rear surface 111b and the first nucleotide.

Substrate 110 may carry on front surface 111a, an identification code, e.g., in the form of bar code (not shown) or the like printed on a substrate in the form of a paper label attached by adhesive or any convenient means. The identification code contains information relating to array 112, where such information may include, but is not limited to, an identification of array 112, i.e., layout information relating to the array(s), etc.

In those embodiments where an array includes two more features immobilized on the same surface of a solid support, the array may be referred to as addressable. An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “probe” may be the one which is to be evaluated by the other (thus, either one could be an unknown mixture of analytes, e.g., polynucleotides, to be evaluated by binding with the other).

A “scan region” refers to a contiguous (preferably, rectangular) area in which the array spots or features of interest, as defined above, are found. The scan region is that portion of the total area illuminated from which the resulting fluorescence is detected and recorded. For the purposes of this invention, the scan region includes the entire area of the slide scanned in each pass of the lens, between the first feature of interest, and the last feature of interest, even if there exist intervening areas which lack features of interest. An “array layout” refers to one or more characteristics of the features, such as feature positioning on the substrate, one or more feature dimensions, and an indication of a moiety at a given location. “Hybridizing” and “binding”, with respect to polynucleotides, are used interchangeably.

The term “substrate” as used herein refers to a surface upon which marker molecules or probes, e.g., an array, may be adhered. Glass slides are the most common substrate for biochips, although fused silica, silicon, plastic and other materials are also suitable.

The term “flexible” is used herein to refer to a structure, e.g., a bottom surface or a cover, that is capable of being bent, folded or similarly manipulated without breakage. For example, a cover is flexible if it is capable of being peeled away from the bottom surface without breakage.

“Flexible” with reference to a substrate or substrate web, references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C.

A “web” references a long continuous piece of substrate material having a length greater than a width. For example, the web length to width ratio may be at least 5/1, 10/1, 50/1, 100/1, 200/1, or 500/1, or even at least 1000/1.

The substrate may be flexible (such as a flexible web). When the substrate is flexible, it may be of various lengths including at least 1 m, at least 2 m, or at least 5 m (or even at least 10 m).

The term “rigid” is used herein to refer to a structure, e.g., a bottom surface or a cover that does not readily bend without breakage, i.e., the structure is not flexible.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

The term “stringent conditions” refers to conditions under which a probe will hybridize. preferentially to its target subsequence, and to a lesser extent to, or not at all to, other sequences. Put another way, the term “stringent hybridization conditions” as used herein refers to conditions that are compatible to produce duplexes on an array surface between complementary binding members, e.g., between probes and complementary targets in a sample, e.g., duplexes of nucleic acid probes, such as DNA probes, and their corresponding nucleic acid targets that are present in the sample, e.g., their corresponding mRNA analytes present in the sample. A “stringent hybridization” and “stringent hybridization wash conditions” in the context of nucleic acid hybridization (e.g., as in array, Southern or Northern hybridizations) are sequence dependent, and are different under different environmental parameters. Stringent hybridization conditions that can be used to identify nucleic acids within the scope of the invention can include, e.g., hybridization in a buffer comprising 50% formamide, 5×SSC, and 1 % SDS at 42° C, or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCI, and 1% SDS at 37° C., and a wash in 1×SSC at 45° C. Alternatively, hybridization to filter-bound DNA in 0.5 M NaHPO₄, 7% sodium dodecyl sulfate (SDS), 1 mnM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions which determine whether a nucleic acid is specifically hybridized to a probe. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. In instances wherein the nucleic acid molecules are deoxyoligonucleotides (“oligos”), stringent conditions can include washing in 6×SSC/0.05% sodium pyrophosphate at 37° C. (for 14-base oligos), 48° C. (for 17-base oligos), 55° C. (for 20-base oligos), and 60° C. (for 23-base oligos). See Sambrook, Ausubel, or Tijssen (cited below) for detailed descriptions of equivalent hybridization and wash conditions and for reagents and buffers, e.g., SSC buffers and equivalent reagents and conditions.

Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed, as appropriate.

By “remote location,” it is meant a location other than the location at which the array is present and hybridization occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. An array “package” may be the array plus only a substrate on which the array is deposited, although the package may include other features (such as a housing with a chamber). A “chamber” references an enclosed volume (although a chamber may be accessible through one or more ports). It will also be appreciated that throughout the present application, that words such as “top,” “upper,” and “lower” are used in a relative sense only.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” references any hardware and/or software combination that will perform the functions required of it. For example, any processor herein may be a programmable digital microprocessor such as available in the form of a electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

### DETAILED DESCRIPTION OF THE INVENTION

Methods of identifying a sequence of a probe, e.g., a biopolymeric probe, such as a nucleic acid, for use as a surface immobilized probe for a target molecule of interest, e.g., a target nucleic acid, are provided. A feature of the subject methods is that a set of candidate sequences is evaluated for full-length synthesis probability, e.g., by evaluating the candidate sequences' depurination susceptibility. The subject invention also includes algorithms for performing the subject methods recorded on a computer readable medium, as well as computational analysis systems that include the same. Also provided are nucleic acid arrays produced with probes having sequences identified by the subject methods, as well as methods for using the same.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described. Methods recited herein may be carried out in any order of the recited events which is logically possible, as well as the recited order of events.

All patents and other references cited in this application, are incorporated into this application by reference except insofar as they may conflict with those of the present application (in which case the present application prevails).

As summarized above, the subject invention provides methods of identifying or designing probes for use in an array structures, where the probes are chemical probes, e.g., biopolymeric probes, such as nucleic acids. While the following description is provided in terms of nucleic acid probe design protocols for ease and clarity of description, the scope of the invention is not so limited, but instead extends to the identification or design of suitable probes for use in any type of array structure.

By way of introduction of the invention, the synthesis protocol used to fabricate an array of biopolymeric probes can have a significant impact on the functional nature probes and features thereof on the array. For example, for in situ array synthesis protocols in which the individual probe nucleic acids of the array features are synthesized directly on a surface of a solid support (as reviewed above), the particular probe synthesis protocol employed can have an impact on the percentage of full length probes that are produced in a given feature. In other words, a given in situ synthesis protocol may produce, in addition to full length probe sequences, non-full length sequences, which non-full length sequences can adversely impact the functionality of the feature.

One reason that non-full length sequences may be produced in addition to desired full length sequences is that in situ produced oligonucleotides are susceptible to depurination side reactions, specifically acid-catalyzed depurination, shown in below in Scheme 1. 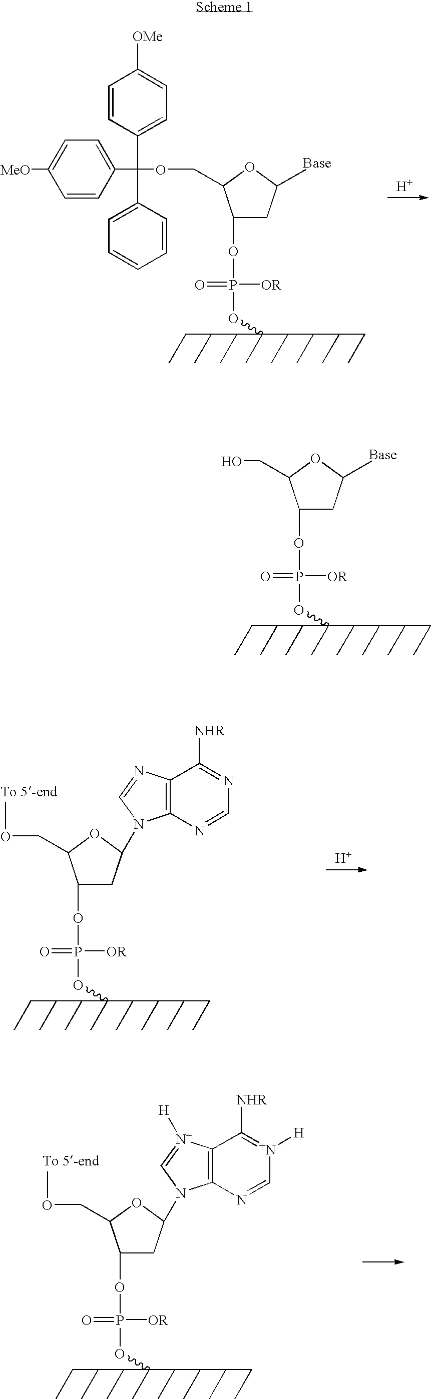 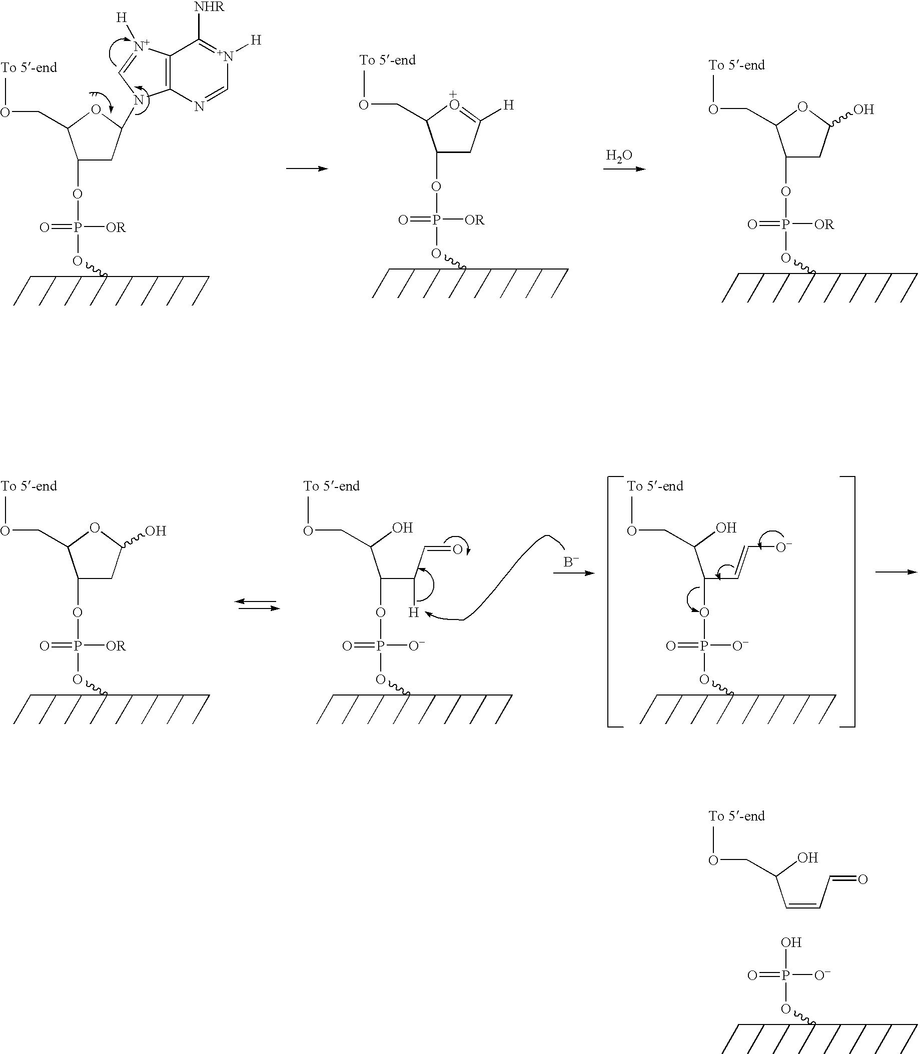
The first line of Scheme 1 shows the desired reaction (deblocking the 5′-hydroxyl at the end of each synthetic cycle) that is responsible for cyclic acid exposure. The second line shows the undesirable, acid-catalyzed side reaction: hydrolysis of the deoxyribose-purine (glycosidic) bond, with conversion of the furan structure of the deoxyribose sugar into an aldose. The base shown in Scheme 1 is adenine (A), because A is by far the more sensitive of the 2 purines. For many embodiments of the application as described below, depurination shall be considered to be strictly a side-reaction of A bases. It should be noted that although G nucleotides are also sensitive to depurination, only the depurination of As are considered in the following representative methods since the depurination rates of Gs are, in practice, negligeable compared to the depurination rates of As in the representative chemistries described below. If other chemistries are used in a given synthesis protocol in which depurination rates of Gs are greater than depurination rates of As, one of skill in the art can readily adapt the following described methods so that the deblock dose is calculated using the sum of deblock reaction experienced by all G nucleotides. Furthermore, if a given protocol provides depurination rates of Gs and As that are similar, the following representative embodiments can be readily modified by those of skill in the art so that both As and Gs are considered in the deblock dose calculation. The final line of Schemel shows the eventual consequences of depurination when the finished oligonucleotide is exposed to a final, base-catalyzed deprotection step to remove protecting groups from the A, C and G bases: the 3′-phosphodiester bond to the aldose sugar is cleaved by β-elimination, cleaving the oligonucleotide backbone, with loss of all bases on the 5′-side of the site of depurination.

Depurination of array-bound oligonucleotides is a particularly pernicious problem, because the oligonucleotides on an in situ-synthesized microarray are not subjected to subsequent purification steps meant to retain only full-length products. Thus, depurination yields a microarray feature that is both depleted in the intended, full-length oligonucleotide and filled with truncated sequences that at best do nothing and at worst degrade the specificity of the full-length probes.

In view of the above, the inventors have realized the desirability of being able to predict the quantitative susceptibility of an array-bound probe to synthesis mediated degradation, such as depurination, during array manufacture, and to incorporate such a prediction at the stage of probe design. The present invention provides methods and compositions designing probes using a prediction of synthesis mediated probe degradation.

In further describing the subject invention the methods for identifying suitable probe sequences are described first in greater detail, followed by a review of arrays that may be produced using probes identified by the subject methods as well as representative applications for such arrays.

Methods

As summarized above, the subject invention provides methods of identifying a sequence of a nucleic acid for use, e.g., that is immediately suitable for use or worthy of further evaluation as suitable for use, as a surface immobilized probe for a target nucleic acid. In other words, the subject invention provides methods of designing nucleic acid probes that can be used on nucleic acid arrays. Specifically, the subject methods result in the identification of one or more probes that are suitable for use (i.e., either directly or after further evaluation) as array probes at least because they have an acceptable probability of being fully synthesized by the synthesis protocol used to fabricate the array on which they appear. In other words, the subject methods identify probe sequences that have an acceptable probability of being fully synthesized by the particular array synthesis protocol that is to be employed.

In many embodiments, the subject methods include the following steps:
(a) identifying a plurality or set of candidate probe sequences for the target nucleic acid;
(b) determining a full length synthesis probability measure for each member sequence of the previously identified set of candidate probe sequences; and
(c) using the determined synthesis probability measures to identify probe sequences of interest, e.g., by selecting those sequences of the set that satisfy a full length synthesis probability threshold to identify at least one sequence of a nucleic acid that is suitable for use as a substrate surface immobilized probe.

Each of the above steps is now reviewed in greater detail below.

Candidate Probe Identification

As mentioned above, the first step in the subject methods is to identify a plurality of candidate probe sequences for a given target nucleic acid of interest. The target nucleic acid of interest is generally a nucleic acid of known sequence, where the length of the nucleic acid may vary, but typically ranges from about 200 nt to about 4,000 nt, such as from about 400 nt to about 2,500 nt, including from about 800 nt to about 1,500 nt. In many embodiments, the target nucleic acid has the sequence of an mRNA transcript of interest or the complementary sequence thereof, or the sequence of a first or second strand DNA prepared from an mRNA of interest.

In this first step of the subject methods, a predetermined number of unique oligonucleotides is identified. The length of the oligonucleotides may be the same or different. The oligonucleotides are unique in that no two of the oligonucleotides are identical. The unique oligonucleotides are chosen to sample the entire length of a nucleotide sequence that is hybridizable with the target nucleotide sequence. The actual number of oligonucleotides is generally determined by the length of the nucleotide sequence and the desired result. In certain embodiments, the number of oligonucleotides is selected to be sufficient to achieve a consensus behavior. In other words, the number of oligonucleotide sequences is sufficiently numerous so that several possible probes overlap or fall within a given region that is expected to yield acceptable hybridization efficiency. Since the location of these regions is not known before hand, one strategy that may be employed is to equally space the probe sequences along the sequence that is hybridizable to the target sequence. Since regions of acceptable hybridization efficiency are generally on the order of 20 nucleotides in length, one practical strategy is to space the starting nucleotides of the oligonucleotide sequences no more than five base pairs apart. If computation time needed to calculate the predictive parameters is not an issue, then one strategy is to space the starting nucleotides one nucleotide apart.

In certain embodiments, a set of overlapping sequences may be chosen. To this end, the subsequences are chosen so that there is overlap of at least one nucleotide from one oligonucleotide to the next. In certain embodiments, the overlap is two or more nucleotides. In certain embodiments, the oligonucleotides are spaced one nucleotide apart and the predetermined number is Y−Z+1 oligonucleotides where Y is the length of the nucleotide sequence and Z is the length of the oligonucleotides. In the latter situation, the unique oligonucleotides are of identical length Z. Thus, a set of overlapping oligonucleotides is a set of oligonucleotides that are subsequences derived from some master sequence by subdividing that sequence in such a way that each subsequence contains either the start or end of at least one other subsequence in the set.

An example of the above for purposes of illustration and not limitation is presented by the sequence ATGGACTTAGCATTCG (SEQ ID NO:01), from which the following set of overlapping oligonucleotides can be identified:

| | |
|---|---|
| ATGGACYVAGCA | (SEQ ID NO:02) |
| TGGACTTAGCAT | (SEQ ID NO:03) |
| GGACTTAGCATT | (SEQ ID NO:04) |
| GACTTAGCATTC | (SEQ ID NO:05) |
| ACTTAGCATTCG | (SEQ ID NO:06) |

In this example the overlapping oligonucleotides are spaced one nucleotide apart. In other words, there is overlap of all but one nucleotide from one oligonucleotide to the next. In the example above, the original nucleotide sequence is 16 nucleotides long (Y=16). The length of each of the overlapping oligonucleotides is 12 nucleotides long (Z=12) and there are 16−12+1=5 oligonucleotides.

The length of the oligonucleotides may be the same or different and may vary depending on the length of the nucleotide sequence. The length of the oligonucleotides is determined by a practical compromise between the limits of current chemistries for oligonucleotide synthesis and the need for longer oligonucleotides, which exhibit greater binding affinity for the target sequence and are more likely to occur only once in complicated mixtures of polynucleotide targets. In certain embodiments, the length of the oligonucleotides is from about 10 to about 100 nucleotides, including from about 10 to about 80 nucleotides, such as from about 25 to about 70 nucleotides.

Using the above protocol, a plurality of candidate probe sequences are identified for a given target nucleic acid. In certain embodiments, the number of identified candidate probe nucleic acid sequences is at least about 5, usually at least about 7 and may be as great as 15, 20 or more, but typically does not exceed about 15, where in certain embodiments, the number of candidate probe sequences identified for a given target nucleic acid ranges from about 7 to 12, e.g., 8, 9, 10or11.

In certain embodiments, an algorithm is employed, e.g., in conjunction with a computational analysis system, to identify candidate probe sequences from a target nucleic acid. Any convenient algorithm or process capable of performing the above function may be employed.

As indicated above, the above first step in the subject methods results in the identification of a plurality of different candidate probe sequences for a given target nucleic acid.

Evaluation of Identified Candidate Probe Nucleic Acid Sequences for Full Length Synthesis Probability

Following provision of the initial set of candidate probe sequences for a given nucleic acid target, as described above, the next step in the subject methods is to evaluate each candidate probe sequence for its full length synthesis probability. By full length synthesis probability is meant the likelihood that a given probe sequence will be fully synthesized using the particular probe synthesis protocol to be employed in manufacturing the array that includes the probes designed according to the subject invention. As such, in this step of the subject methods, a measure or determination is made which serves as a prediction of whether or not, based on the sequence of residues in the candidate probe sequence, the probe will be fully synthesized by the to be employed array synthesis protocol.

Where the to be employed array synthesis protocol is an in situ array synthesis protocol, as described above, certain embodiments of interest determine or predict the propensity of a candidate probe of a given sequence to suffer from depurination during synthesis of the probe. In other words, an evaluation of a probe's susceptibility to depurination during synthesis is made based on the sequence of the candidate probe.

In such embodiments, any convenient protocol may be employed to determine or evaluate, i.e., assess or otherwise measure, a candidate probe's susceptibility to depurination during synthesis. In certain embodiments, the protocol employed measures depurination susceptibility by determining the total “deblock” dose of the candidate probe. By total deblock dose is meant the sum of individual deblock doses over all purines, and particularly over all A nucleotides, in positions of the candidate probe sequence where depurination would markedly affect that probe's hybridization performance. For example, in many embodiments A nucleotides at every position except for that at the 5′-terminus are counted when calculating total deblock dose. In other words, the total deblock dose is the sum of all individual deblock doses for each purine, and particular each A, residue in the candidate probe sequence, but for the 5′ terminal residue.

Any given A residue's individual deblock dose is the total number of deblock cycle exposures experienced by that nucleotide during array manufacture. As such, the general formula for deblock dose d(x) for an A nucleotide written at layer x of an array synthesized by a process having L total layers is
d(x)=L−x+1 (Eq. 1)

Therefore, the overall deblock dose for a sequence containing N A nucleotides written at layers x₁, x₂, . . . , x_{N} during an in situ synthesis protocol is

$$\begin{matrix}{D}_{Total} = {\sum }_{i = 1}^{N} d ( {x}_{i} ) \\ = N ( L + 1 ) - {\sum }_{i = 1}^{N} {x}_{i}\end{matrix} \qquad ( Eq . 2 )$$

The above step of the subject methods results in each candidate sequence of the initially identified set being evaluated or measured for its probability of full-length synthesis, e.g., by determination of its depurination susceptibility (such as measured by determining its total deblock dose). The measurements or evaluations obtained for each of the candidate probe sequences in this substep of the subject methods are then employed in the next substep to identify those members of the initial set that are suitable for use on an array, at least in terms of their full length synthesis probability.

Selection of Optimum Candidate Probe Sequence

In the final step of the subject methods, a probe sequence that can be employed in a probe suitable for use as a surface immobilized probe for the target of interest is selected from the full-length synthesis probability evaluated candidate sequences of the previous step, specifically by using the full length synthesis probability measures as determined in the previous step. In other words, any probe sequences having desirable full-length synthesis probabilities, as determined in the above evaluation step, are selected for further use, e.g., as a probe sequence on an array or further evaluation of suitability for probe use on an array. Typically, probe sequences are selected from the evaluated candidate probe sequences by selecting those probe sequence that satisfy a predetermined full-length synthesis probability threshold.

In those embodiments where full-length synthesis probability is evaluated in terms of deblock dose, those sequences that satisfy a predetermined deblock dose threshold are chosen or selected. In many embodiments, sequences that satisfy a predetermined deblock dose threshold are ones that have a measured or evaluated (i.e., determined) deblock dose that does not exceed a predetermined deblock dose threshold. The deblock dose threshold is typically less than 50% of the maximum deblock dose based (determined based on length of the probe) where in certain embodiments it may be from about 10% to about 50%, such as from about 20% to about 45% of the maximum deblock dose. More specifically, a maxium deblock dose depends upon L (the maximum deblock dose is L(L+1)/2). So, for 60-mers, the maximum dose is 1830. For such probes, a representative threshold would be one between about 22% (conservative) and 44% (permissive) of that maximal dose of 1830.

The above described methodology results in the selection of probe sequences for use in surface immobilized probes that have an acceptably sufficient probability of being fully synthesized by the synthesis method that is employed to produce the array that contains the designed sequence. In other words, the subject methods identify probe sequences that have an acceptably low susceptibility to degradation, e.g., via depurination, during their synthesis. As such, the subject methods identify sequences that are likely to have a high average single step yield, e.g., a single step yield of at least about 95%, such as at least about 98%, including at least about 99% or greater.

In many embodiments, the probe nucleic acid sequences identified using the subject methods are provided in text format or as a string of text, where the text represents or corresponds to the sequence of nucleotides of a probe nucleic acid. The nucleic acid sequences can be of any length, where the nucleic acid sequences are typically about 20 nt to about 100 nt in length, e.g., from about 20 to about 80 nt in length, e.g., 25 nt, 60 nt, etc. However, nucleic acid sequences of lesser or greater length may be identified as appropriate. Suitable nucleic acid probes produced therefrom may be oligonucleotides or polynucleotides, as will be described in greater detail below.

Optional Additional Steps

The above described probe design process that includes the evaluation of full length probe synthesis probability may be incorporated into an overall probe design protocol that evaluates candidate probes for one or more additional parameters. As such, in certain embodiments, candidate probes are identified based on a least one functionally relevant selection criterion (i.e., a selection criteria or parameter that is known or believe to have an impact on the functionality of the probe), where in certain embodiments a plurality of different functionally relevant selection criteria may be employed together to identify desirable probe sequences, where by plurality is meant at least about 2, and may be as greater as 10 or more, but is typically less than 5, e.g., 2 to 3. Functionally relevant criteria or parameters include, but are not limited to: thermodynamic characteristics, base composition, self-structure, and homology to non-target sequences likely to present in samples of interest, e.g., as described in U.S. Pat. No. 6,251,588, the disclosure of which is herein incorporated by reference.

One functionally relevant selection criterion of interest that may be employed is distance from the 3′-end of the mRNA transcript that corresponds to the target nucleic acid, e.g., that is the target nucleic acid or is the complement of the target nucleic acid, or from which the target nucleic acid is derived, e.g., where the target nucleic acid is first or second strand cDNA. When this criterion is employed, candidate sequences of the target nucleic acid are chosen that are within at least about 2,000 nt, usually within about 1,500 nt and more usually within about 800 nt of the 3′ end of the mRNA that corresponds to the target nucleic acid.

Another functionally relevant selection criterion of interest is the base composition of the probe sequence. When this criterion is employed, sequences that are abnormally GC rich or poor, long runs of a single base, and/or base compositions that are known to generate unacceptable array features, e.g., under in situ production conditions are avoided. Sequences that are abnormally GC rich or poor are those sequences whose number % of G and C bases are greater than about 30, such as greater than about 35, or less than about 60, such as less than about 45. By “long run” of a single base is meant a stretch of nucleotides of the same base that is greater than about 6, such as greater than about 10. Sequences that are known to generate unacceptable array features include, but are not limited to those containing stretches of at least 10 Gs.

Another functionally relevant selection criterion of interest is homology of the candidate probe sequence to other sequences from the same organism, i.e., to other mRNA transcripts or complements thereof of the same organism from which the target sequence of interest for which the probe is being designed is obtained. Sequences with a high potential to hybridize to more than one mRNA transcript from a given organism are avoided. Cross-hybridization potential of candidate sequences may be estimated via thermodynamic scoring of the output of BLAST, a standard bioinformatics application used to detect sequence homology and well known to those of skill in the art, or any other convenient cross-hybridization potential assessment protocol. Use of this criterion results in the identification of probe sequences that are specific for the target nucleic acid of interest.

The above reviewed functionally relevant criteria are merely representative of the different criteria or parameters that may be used in certain embodiments to identify an initial set of candidate probe sequences.

Depending on the particular probe design protocol, the subject full length synthesis evaluation step may be practiced at the beginning, end or middle of the overall probe design protocol. Many probe design algorithms or protocols operate by subtraction: all possible probes that meet a minimal set of length and position requirements are constructed, and probes are then subsequently removed via a set of filters. The probes that survive the filtration process are assigned ranks based on some combination of the filtered metrics, and the probes with the highest predicted quality are chosen for experimental testing. A major improvement to this basic scheme clusters probes that pass the metric filters by proximity on the target sequence, and assigns better scores to larger clusters (see U.S. Pat. No. 6,251,588; the disclosure of which is herein incorporated by reference).

In overall probe design protocols, the full length synthesis evaluation, e.g., in the form of predicted deblock dose, can be used to filter probes at either the beginning or end of the probe design process. For example, filtering away probes with high deblock doses is most effective at the beginning of a probe design process that does not employ clustering, since deblock dose is relatively easy to compute, and early filtering will prevent the design algorithm from performing more intensive calculations on probes with high susceptibilities to depurination. Minimization of deblock dose is most effective at the end of an algorithm that considers probe clustering, since the deblock doses of probes that are clustered (adjacent) along the target sequence are not independent, and the main effect of consideration of deblock dose will be to indicate which of several probes in a cluster is the most robust choice.

Computer Programming

One or more aspects of the above methodology may be in the form of computer readable media having programming stored thereon for implementing the subject methods. The computer readable media may be, for example, in the form of a computer disk or CD, a floppy disc, a magnetic “hard card”, a server, or any other computer readable media capable of containing data or the like, stored electronically, magnetically, optically or by other means. Accordingly, stored programming embodying steps for carrying-out the subject methods may be transferred to a computer such as a personal computer (PC), (i.e., accessible by a researcher or the like), by physical transfer of a CD, floppy disk, or like medium, or may be transferred using a computer network, server, or other interface connection, e.g., the Internet.

Computational Analysis System

In one embodiment of the subject invention, a system of the invention may include a single computer or the like with a stored algorithm capable of carrying out suitable probe identification methods, i.e., a computational analysis system. In certain embodiments, the system is further characterized in that it provides a user interface, where the user interface presents to a user the option of selecting among one or more different, including multiple different, inputs, e.g., various parameter values for the algorithm, as described above, such as distance from 3′ end, definition of overlap, t, etc. Computational systems that may be readily modified to become systems of the subject invention include those described in U.S. Pat. No. 6,251,588; the disclosure of which is herein incorporated by reference.

Utility

The above-described methods, as well as devices programmed to practice the same, may be used to identify probe nucleic acids to be produced on surfaces of any of a variety of different substrates, including both flexible and rigid substrates, e.g., in the production of nucleic acid arrays. Preferred materials provide physical support for the deposited material and endure the conditions of the deposition process and of any subsequent treatment or handling or processing that may be encountered in the use of the particular array. The array substrate may take any of a variety of configurations ranging from simple to complex. Thus, the substrate could have generally planar form, as for example, a slide or plate configuration, such as a rectangular or square disc. In many embodiments, the substrate will be shaped generally as a rectangular solid, having a length in the range of about 4 mm to 200 mm, usually about 4 mm to 150 mm, more usually about 4 mm to 125 mm; a width in the range of about 4 mm to 200 mm, usually 30 about 4 mm to 120 mm, and more usually about 4 mm to about 80 mm; and a thickness in the range of about 0.01 mm to about 5 mm, usually from about 0.1 mm to about 2 mm and more usually from about 0.2 mm to about 1 mm. However, larger or smaller substrates may be and can be used, particularly when such are cut after fabrication into smaller size substrates carrying a smaller total number of arrays 12. Substrates of other configurations and equivalent areas can be chosen. The configuration of the array may be selected according to manufacturing, handling, and use considerations.

The substrates may be fabricated from any of a variety of materials. In certain embodiments, such as for example where production of binding pair arrays for use in research and related applications is desired, the materials from which the substrate may be fabricated should ideally exhibit a low level of non-specific binding during hybridization events. In many situations, it will also be preferable to employ a material that is transparent to visible and/or UV light. For flexible substrates, materials of interest include: nylon, both modified and unmodified, nitrocellulose, polypropylene, and the like, where a nylon membrane, as well as derivatives thereof, may be particularly useful in this embodiment. For rigid substrates, specific materials of interest include: glass; fuse silica; silicon, plastics (for example polytetraflouroethylene, polypropylene, polystyrene, polycarbonate, and blends thereof, and the like); metals (for example, gold, platinum, and the like).

The substrate surface onto which the polynucleotide compositions or other moieties are deposited may be smooth or substantially planar, or have irregularities, such as depressions or elevations. The surface may be modified with one or more different layers of compounds that serve to modify the properties of the surface in a desirable manner. Such modification layers of interest include: inorganic and organic layers such as metals, metal oxides, polymers, small organic molecules and the like. Polymeric layers of interest include layers of: peptides, proteins, polynucleic acids or mimetics thereof (for example, peptide nucleic acids and the like); polysaccharides, phospholipids, polyurethanes, polyesters, polycarbonates, polyureas, polyamides, polyetheyleneamines, polyarylene sulfides, polysiloxanes, polyimides, polyacetates, and the like, where the polymers may be hetero- or homopolymeric, and may or may not have separate functional moieties attached thereto (for example, conjugated).

Arrays

Also provided by the subject invention are novel nucleic acid arrays of produced using the subject methods, as described above. The subject arrays include at one probe, and typically a plurality of different probes of different sequence (e.g., at least about 10, usually at least about 50, such as at least about 100, 1000, 5000, 10,000 or more) immobilized on, e.g., covalently or non-covalently attached to, different and known locations on the substrate surface. A feature of the subject arrays is that at least one of the probes is a probe having a sequence identified according to the present methods, where in many embodiments at least about 5, 10, 50, 100, 500, 1000, 5000, 10000 or more of the probe sequences are sequences identified by the subject methods. Each distinct nucleic acid sequence of the array is typically present as a composition of multiple copies of the polymer on the substrate surface, e.g. as a spot on the surface of the substrate. The number of distinct nucleic acid sequences, and hence spots or similar structures (i.e., array features), present on the array may vary, but is generally at least 2, usually at least 5 and more usually at least 10, where the number of different spots on the array may be as a high as 50, 100, 500, 1000, 10,000 or higher, depending on the intended use of the array. The spots of distinct nucleic acids present on the array surface are generally present as a pattern, where the pattern may be in the form of organized rows and columns of spots, e.g., a grid of spots, across the substrate surface, a series of curvilinear rows across the substrate surface, e.g., a series of concentric circles or semi-circles of spots, and the like. The density of spots present on the array surface may vary, but will generally be at least about 10 and usually at least about 100 spots/cm², where the density may be as high as 10⁶ or higher, but will generally not exceed about 10⁵ spots/cm². In the subject arrays of nucleic acids, the nucleic acids may be covalently attached to the arrays at any point along the nucleic acid chain, but are generally attached at one of their termini, e.g., the 3′ or 5′ terminus.

A feature of the subject arrays is that they include one or more, usually a plurality of, probes whose sequence as been selected according to the subject protocols. Because the sequences of the probes on the arrays are selected according to the above protocols, the probe sequences are ones that are likely to be produced in full length via the synthesis protocol employed to make the array. As such, as described above, the features of the array are ones that have a high proportion of full length sequences, and substantially little, if any, non-full length sequences.

Utility of Arrays

The subject arrays find use in a variety applications, where such applications are generally analyte detection applications in which the presence of a particular analyte in a given sample is detected at least qualitatively, if not quantitatively. Protocols for carrying out such assays are well known to those of skill in the art and need not be described in great detail here. Generally, the sample suspected of comprising the analyte of interest is contacted with an array produced according to the subject methods under conditions sufficient for the analyte to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g. through use of a signal production system, e.g., an isotopic or fluorescent label present on the analyte, etc. The presence of the analyte in the sample is then deduced from the detection of binding complexes on the substrate surface.

Specific analyte detection applications of interest include hybridization assays in which the nucleic acid arrays of the subject invention are employed. In these assays, a sample of target nucleic acids is first prepared, where preparation may include labeling of the target nucleic acids with a label, e.g., a member of signal producing system. Where the arrays include “all-bases-all-layers” control probes, as described above, a collection of labeled control targets is typically included in the sample, where the collection may be made up of control targets that are all labeled with the same label or two or more sets that are distinguishably labeled with different labels, as described above. Following sample preparation, the sample is contacted with the array under hybridization conditions, whereby complexes are formed between target nucleic acids that are complementary to probe sequences attached to the array surface. The presence of hybridized complexes is then detected. Specific hybridization assays of interest which may be practiced using the subject arrays include: gene discovery assays, differential gene expression analysis assays; nucleic acid sequencing assays, and the like. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

In certain embodiments, the subject methods include a step of transmitting data from at least one of the detecting and deriving steps, as described above, to a remote location. By “remote location” is meant a location other than the location at which the array is present and hybridization occur. For example, a remote location could be another location (e.g., office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

As such, in using an array made by the method of the present invention, the array will typically be exposed to a sample (for example, a fluorescently labeled analyte, e.g., protein containing sample) and the array then read. Reading of the array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER device available from Agilent Technologies, Palo Alto, Calif. Other suitable apparatus and methods are described in U.S. Pat. Nos. 5,091,652; 5,260,578; 5,296,700; 5,324,633; 5,585,639; 5,760,951; 5,763,870; 6,084,991; 6,222,664; 6,284,465; 6,371,370 6,320,196 and 6,355,934; the disclosures of which are herein incorporated by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample). The results of the reading (processed or not) may be forwarded (such as by communication) to a remote location if desired, and received there for further use (such as further processing).

Kits

Kits for use in analyte detection assays are also provided. The kits at least include the arrays of the invention, as described above. The kits may further include one or more additional components necessary for carrying out an analyte detection assay, such as sample preparation reagents, buffers, labels, and the like. As such, the kits may include one or more containers such as vials or bottles, with each container containing a separate component for the assay, and reagents for carrying out an array assay such as a nucleic acid hybridization assay or the like. The kits may also include a denaturation reagent for denaturing the analyte, buffers such as hybridization buffers, wash mediums, enzyme substrates, reagents for generating a labeled target sample such as a labeled target nucleic acid sample, negative and positive controls and written instructions for using the array assay devices for carrying out an array based assay. Such kits also typically include instructions for use in practicing array based assays.

Kits for use in connection with the probe design protocols of the subject invention may also be provided. Such kits may include at least a computer readable medium including programming as discussed above and instructions. The instructions may include installation or setup directions. The instructions may include directions for use of the invention.

Providing software and instructions as a kit may serve a number of purposes. The combinations may be packaged and purchased as a means of upgrading an existing fabrication device. Alternatively, the combination may be provided in connection with a new device for fabricating arrays, in which the software may be preloaded on the same. In which case, the instructions will serve as a reference manual (or a part thereof and the computer readable medium as a backup copy to the preloaded utility.

The instructions of the above-described kits are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or sub packaging), etc. In other embodiments, the instructions are present as an electronic storage data file present on a suitable computer readable storage medium, e.g., CD-ROM, diskette, etc, including the same medium on which the program is presented.

In yet other embodiments, the instructions are not themselves present in the kit, but means for obtaining the instructions from a remote source, e.g. via the Internet, are provided. An example of this embodiment is a kit that includes a web address where the instructions can be viewed and/or from which the instructions can be downloaded. Conversely, means may be provided for obtaining the subject programming from a remote source, such as by providing a web address. Still further, the kit may be one in which both the instructions and software are obtained or downloaded from a remote source, as in the Internet or World Wide Web. Some form of access security or identification protocol may be used to limit access to those entitled to use the subject invention. As with the instructions, the means for obtaining the instructions and/or programming is generally recorded on a suitable recording medium.

The following examples are offered by way of illustration and not by way of limitation.

### Experimental

I. Modeling Depurination

The effect of depurination on the signal generated by a probe feature after hybridization to a given target can be modeled via the separate components of signal. The general model can be written as
S=S_{B}+Hc_{target}T(λ)Y(λ+m)Q_{intact}, (Eq. 3)
where S is the observed signal, S_{B} is the background signal, H is a constant that includes both instrument and intrinsic probe hybridization efficiency effects, c_{target} is the hybridization target concentration, T(λ) is the tether enhancement for a tether of length λ, Y(λ+m) is the full-length oligonucleotide yield after λ+m layers (λ for the tether, m for the hybridizing probe) and Q_{intact} is the probability that the probe has not depurinated at any A nucleotide (“survival probability”). The background term is assumed to be small and relatively independent of the other probe parameters (i.e. it will be a simple additive constant in the final model). Equation 3 implies that the depurination survival probability Q_{intact} contributes to probe signal in the same way as other parameters used to pick good probes. For example, probe/target duplex thermodynamics and probe self-structure help determine the constant H, while chemosynthetic efficiency determines the function Y(λ+m); both of these parameters have been used to design probes.
II. Survival Probability:

The probe survival probability Q_{intact} can be modeled in a straightforward fashion with one assumption: depurination behaves as a pseudo 1^{st}-order reaction. Given this assumption and some standard chemical kinetics, the probability pᵢ that a given A nucleotide depurinates during the iₜₕ deblock exposure (which has duration Δtᵢ) is given by
pᵢ=1−e^{kΔt}^{ᵢ}, (Eq. 4)
where k is the pseudo 1^{st}-order rate constant for the depurination reaction. The rate constant k is generally a function of the acid concentration, solvent, temperature, etc.; for the purposes of this disclosure, it is assumed to be the same for all cycles. Note however that depurination rate could depend upon distance from the surface (i.e. it might not be the same for A's in different positions in an oligo). However, the effect of a change in k is exactly the same as the effect of the same percent change in Δtᵢ. Therefore, the model suffers no formal loss of generality, so long as it allows different depurination probabilities pᵢ for different deblock exposures.

The probability that a given A nucleotide survives the i^{th} deblock exposure is simply
qᵢ=1−pᵢ (Eq. 5)
and the probability that a given A at position x survives all of the deblock exposures it experiences is

$$q ( x ) = {\prod }_{all relevant i} {q}_{i} . \qquad ( Eq . 6 )$$

If the probability pᵢ for all A's at all exposures has the same value p for all values of i, then it is easy to show that
q(x)=(1−p)^{d(x)} (Eq. 7).
where d(x) is the deblock dose experienced by the A nucleotide at position x; d(x) is given by Eq. 1. The overall survival probability Q_{intact} is simply the product over all relevant values of x of the individual survival probabilities q(x):

$$\begin{matrix} {Q}_{intact} = {\prod }_{all relevant x} {( 1 - p )}^{d ( x )} \\ ⇒ & ( Eq . 8 ) \\ log ( {Q}_{intact} ) = [ {\sum }_{all relevant x} d ( x ) ] log ( 1 - p ) \\ ≡ {D}_{Total} log ( 1 - p ) \\ ⇒ \\ {Q}_{intact} = {( 1 - p )}^{{D}_{Total}} & \end{matrix}$$

where Dₜₒₜₐₗ is given by Eq. 2.

Equations 1-8 together state that there is a straightforward, calculable set of relationships between probe sequence, probe depurination susceptibility and observed probe performance. This set of relationships can be used to design probes that exhibit minimal susceptibility to depurination.

III. Deblock Dose

Probe susceptibilities to depurination can be predicted by introducing the concept of “deblock dose”. The deblock dose of a given A nucleotide is the total number of deblock cycle exposures experienced by that nucleotide during array manufacture. The total deblock dose of a given depurination probe is the sum of individual deblock doses over all A nucleotides in positions where depurination would markedly affect that probe's hybridization performance. For the purposes of this disclosure, every position except for that at the 5′-terminus shall be counted when calculating total deblock dose¹. A Visual Basic function for generalized calculation of deblock dose is included in Section IV, below.

The general formula for deblock dose d(x) for an A nucleotide written at layer x of an array with L total layers is
d(x)=L−x+1 (Eq. 1)

¹We know from experimental measurement that deletion of the 5′-A nucleotide from Pro25G decreases the resulting hybridization intensity by less that 10%.

Therefore, the overall deblock dose for a sequence containing N A nucleotides written at layers x₁, x₂, . . . , x_{N} is

$$\begin{matrix}{D}_{Total} = {\sum }_{i = 1}^{N} d ( {x}_{i} ) \\ = N ( L + 1 ) - {\sum }_{i = 1}^{N} {x}_{i}\end{matrix} \qquad ( Eq . 2 )$$

Deblock dose can be used to calculate the fraction of probe that escapes depurination. Deblock doses for several sequences are shown in Table 1; deblock dose D has been calculated assuming a 60-layer array. Skip “_” characters are shown at the 3′ end of the second sequence, indicating that the first base of this sequence is not written until layer 31.

TABLE 1
Deblock Doses for Several Sequences

| | | | |
|---|---|---|---|
| Name | Sequence (5′ to 3′) | Length | D² |

| | | | |
|---|---|---|---|
| Pro25G | ATCATCGTAGCTGGTCAGTGTATCC | 25 | 192 |
| | (SEQ ID NO:07) | | |
| Pro25G_B30 | ATCATCGTAGCTGGTCAGTGTATCC________________ | 25 | 72 |
| | (SEQ ID NO:08) | | |
| Pro25G_DT_A_10_3P | ATCATCGTAGCTGGTCAGTGTATCCAAAAAAAAAA | 35 | 707 |
| | (SEQ ID NO:09) | | |

²Not including the 5′ A nucleotide.

IV. Algorithm I

Visual Basic code for calculation of deblock dose:

′Calculate Deblock Dose, with option of omitting 5′-A from calculation,
′since depurination at this position minimally impacts hyb signal.
′Sequence is assumed to be provided 5′ to 3′, with 3′ skip (“_”)
′characters to indicate skipped layers; 5′-skip characters are
′also permitted, but ignored, since they do not affect deblock dose.

Dim I As Long
Dim N As Long
Dim Noriginal As Long
Dim Acount As Long
Dim aBase As String
DeblockDose2 = 0 ′default
theSequence = UCase(Trim(theSequence)) ′make sequence unambiguous
N = Len(theSequence)
Noriginal = N
′correct for 5′ skip characters
For I = 1 To Noriginal

If Mid(theSequence, I, 1) = “_” Then

N = N − 1

Else

Exit For

End If

Next I
′MsgBox “N = ” & N
Acount = 0
If N > tLayers Then

DeblockDose2 = “Illegal Sequence”
Exit Function

End If
For I = 1 To tLayers

If (I <= N And Not omit5PrimeA) Or (I < N) Then

aBase = Mid(theSequence, Noriginal − I + 1,1)
If aBase = “A” Then Acount = Acount + 1 ′this A contributes from this layer on

End If
DeblockDose2 = DeblockDose2 + Acount ′add contribution from this layer

Next I

End Function

As reviewed above, the subject invention provides methods of identifying probes for use on nucleic acid arrays. However, the subject invention can be used with a number of different types of arrays in which a plurality of distinct polymeric binding agents (i.e., of differing sequence) are stably associated with (i.e., immobilized on) at least one surface of a substrate or solid support by a step-wise synthesis protocol. As such, the polymeric binding agents may vary widely, however polymeric binding agents of particular interest include peptides, proteins, nucleic acids, polysaccharides, synthetic mimetics of such biopolymeric binding agents, etc. In many embodiments of interest, the biopolymeric arrays are arrays of nucleic acids, including oligonucleotides, polynucleotides, cDNAs, mRNAs, synthetic mimetics thereof, and the like.

As such, while the subject methods and devices find use in producing nucleic acid arrays (as described above), the subject devices also find use in the production of non-nucleic acid ligand arrays in which a step-wise or in situ synthesis approach is employed. That is, any of a number of different types of ligand arrays may be produced by the methods of the subject invention, where a first member of a binding pair, typically referred to herein as the ligand is stably associated with the surface of a substrate. For ease of description only, the subject methods and devices described above were described primarily in reference to nucleic acid arrays, where such examples are not intended to limit the scope of the invention. It will be appreciated by those of skill in the art that the subject devices and methods may be employed for use in the production of other types of ligand arrays, e.g., peptide arrays etc., where the ligands of arrays may be synthesized using a step-wise synthesis protocol, particularly where a degradation side reaction may occur in the employed step-wise synthesis protocol.

It is evident from the above results and discussion that a new and useful method of designing probes for use on nucleic acid microarrays is provided by the subject invention. The invention provides methods for the prediction of full-length probe synthesis, e.g., in the form of calculation of deblock dose, a parameter that can be directly related to the intrinsic susceptibility of the probe to depurination during array manufacture. The invention provides methods for the use of full length synthesis probability (e.g., in the form of deblock dose or any quantity derived from deblock dose) as an indicator of probe quality during probe design. The invention provides methods for designing more robust oligonucleotide arrays. As such, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method of identifying a sequence of a nucleic acid for use as a substrate surface immobilized probe for a target nucleic acid, said method comprising:
(a) determining a full length synthesis probability measure for each member sequence of a set of a plurality of candidate probe sequences for said target nucleic acid; and
(b) employing said determined full length synthesis probability measures to select a sequence for use as a substrate immobilized probe for said target nucleic acid.

2. The method according to claim 1, wherein those sequences of said set that satisfy a full length synthesis probability threshold are selected.

3. The method according to claim 2, wherein said full length synthesis probability measure is a measure of the probability that said candidate probe sequence will be fully synthesized by an in situ nucleic acid synthesis protocol.

4. The method according to claim 3, wherein said full length synthesis probability measure is an evaluation of said candidate probe sequence's depurination susceptibility during said in situ nucleic acid synthesis protocol.

5. The method according to claim 4, wherein said depurination susceptibility measure is a deblock dose or parameter based thereon.

6. The method according to claim 5, wherein said method comprises determining a total deblock dose for each member sequence of said set.

7. The method according to claim 6, wherein said full length synthesis probability threshold is a total deblock dose threshold and selected sequences have a total deblock dose that does not exceed said deblock dose threshold.

8. The method according to claim 6, wherein said total deblock dose is a sum of individual deblock doses over all A nucleotides of said candidate probe sequence, except for any 5′ terminal A nucleotide.

9. The method according to claim 8, wherein each of said individual deblock doses d(x) for an A nucleotide positioned at layer x of a candidate probe sequence to be produced by a process having L total layers is equal to L−x+1.

10. The method according to claim 1, wherein at least some of said steps are carried out by a computational analysis system.

11. A computer-readable medium having recorded thereon a program that identifies a sequence of a nucleic acid that is suitable for use as a substrate surface immobilized probe for a target nucleic acid according to the method of claim 1.

12. A computational analysis system comprising a computer-readable medium according to claim 11.

13. A method of producing a nucleic acid array, said method comprising:
producing at least two different probe nucleic acids immobilized on a surface of a solid support, wherein at least one of said at least two different probe nucleic acids has a sequence of nucleotides identified according to the method of claim 1.

14. The method according to claim 13, wherein said at least two different probe nucleic acids are produced on said surface of said solid support by synthesizing said probe nucleic acids on said surface.

15. The method according to claim 13, wherein said at least two different probe nucleic acids are produced on said surface of said solid support by depositing said at least two different probe nucleic acids onto said surface of said solid support.

16. A nucleic acid array produced according to the method of claim 13.

17. A method of detecting the presence of a nucleic acid analyte in a sample, said method comprising:
(a) contacting a nucleic acid array according to claim 16 having a nucleic acid probe that specifically binds to said nucleic acid analyte with a sample suspected of comprising said analyte under conditions sufficient for binding of said analyte to said nucleic acid ligand on said array to occur; and
(b) detecting the presence of binding complexes on the surface of said array to detect the presence of said analyte in said sample.

18. A method comprising transmitting a result from a reading of an array according to the method of claim 17 from a first location to a second location.

19. The method according to claim 18, wherein said second location is a remote location.

20. A method comprising receiving a transmitted result of a reading of an array obtained according to the method claim 17.

21. A kit for identifying a sequence of a nucleic acid that is suitable for use as a substrate surface immobilized probe for a target nucleic acid, said kit comprising:
(a) an algorithm that identifies a sequence of a nucleic acid that is suitable for use as a substrate surface immobilized probe for said target nucleic acid according to the method according to claim 1, wherein said algorithm is present on a computer readable medium; and
(b) instructions for using said algorithm to identify said sequence of a nucleic acid that is suitable for use as a substrate surface immobilized probe for said target nucleic acid.

22. In a method of identifying a sequence of a nucleic acid that is suitable for use as a substrate surface immobilized probe for a target nucleic acid, the improvement comprising:
evaluating a plurality of candidate probe sequences for said target nucleic acid for depurination susceptibility.

23. In a method of identifying a sequence of a polymeric ligand for use as a substrate surface immobilized probe, the improvement comprising:
evaluating a plurality of candidate probe sequences for said polymeric ligand for synthesis degradation probability.
